(19) United States
(12) Patent Application Publication
(10) Pub. No.: US 2022/0313071 A1
(43) Pub. Date: Oct. 6, 2022

(54) MEDICAL OVERTUBE

(71) Applicant: OLYMPUS CORPORATION, Tokyo (JP)

(72) Inventor: Tatsuya HIGUCHI, Tokyo (JP)

(73) Assignee: OLYMPUS CORPORATION, Tokyo (JP)

(21) Appl. No.: 17/842,168

(22) Filed: Jun. 16, 2022

Related U.S. Application Data
(63) Continuation of application No. PCT/JP2019/050590, filed on Dec. 24, 2019.

(51) Int. Cl. A61B 1/00; A61M 25/00

(52) U.S. Cl. CPC A61B 1/00154; A61M 25/0054; A61B 1/00082

(57) ABSTRACT

A medical overtube to be inserted into a body cavity, the medical overtube including a flexible elongated part having a channel through which an elongated medical device is inserted, and a tubular distal end portion connected to a distal end of the elongated part. The distal end portion includes a first section having an end opening which forms an opening of the channel, and a second section located next to a base-end side of the first section, and the medical overtube satisfies the Conditional Expressions K1>K2 and K3>K2, where K1 is rigidity of the first section, K2 is rigidity of the second section, and K3 is rigidity of the elongated part.

### CROSS-REFERENCE TO RELATED APPLICATIONS

This is a continuation of International Application PCT/JP2019/050590, with an international filing date of Dec. 24, 2019, which is hereby incorporated by reference herein in its entirety.

### TECHNICAL FIELD

The present invention relates to a medical overtube.

### BACKGROUND ART

A known overtube through which an endoscope is inserted is configured such that a distal end portion having an opening, through which the endoscope is projected and retracted, has lower strength than a portion located on the base-end side thereof (for example, see PTL 1).

### CITATION LIST

### Patent Literature

{PTL 1} Japanese Unexamined Patent Application Publication No. 2015-157060

### SUMMARY OF INVENTION

An aspect of the present invention is a medical overtube to be inserted into a body cavity, the medical overtube including: a flexible elongated part having a channel through which an elongated medical device is inserted; and a tubular distal end portion connected to a distal end of the elongated part, wherein the distal end portion includes a first section having an end opening which forms an opening of the channel, and a second section located next to a base-end side of the first section, and the medical overtube satisfies the following Conditional Expressions:

K1>K2; and

K3>K2,

wherein K1 is rigidity of the first section, K2 is rigidity of the second section, and K3 is rigidity of the elongated part.

### BRIEF DESCRIPTION OF DRAWINGS

FIG. 1 is a perspective view showing a medical overtube according to an embodiment of the present invention.

FIG. 2 is a cross-section of a main tube of the medical overtube in FIG. 1.

FIG. 3 is a partial longitudinal sectional view for explaining the shape of an end tip of the medical overtube in FIG. 1.

FIG. 4 is a diagram for explaining an example of treatment using the medical overtube in FIG. 1 and schematically shows a state in which the endoscope is inserted into the large intestine.

FIG. 5 schematically shows a process of inserting the medical overtube from the state in FIG. 4.

FIG. 6 schematically shows a state in which a balloon of the medical overtube inserted in FIG. 5 is expanded.

FIG. 7 is a schematic view for explaining a state in which treatment is performed while the endoscope is stably operated with respect to the medical overtube fixed to the large intestine in the state in FIG. 6.

FIG. 8 is a partial longitudinal sectional view for explaining a behavior when the end tip in FIG. 3 goes past a bending section of an insertion section of the endoscope.

FIG. 9 is a partial longitudinal sectional view for explaining a modification of the end tip in FIG. 3.

FIG. 10 is a partial longitudinal sectional view for explaining the operation of the end tip in FIG. 9.

FIG. 11 is a partial enlarged view showing a modification of a fixing mechanism of the medical overtube in FIG. 1.

FIG. 12 is a partial enlarged view for explaining the operation of the medical overtube in FIG. 11.

FIG. 13 is a partial enlarged view showing another modification of the medical overtube in FIG. 1.

FIG. 14 is a partial enlarged view showing another modification of the medical overtube in FIG. 1.

FIG. 15 is a partial enlarged view showing a state in which a balloon of the medical overtube in FIG. 14 is expanded.

### DESCRIPTION OF EMBODIMENTS

A medical overtube 1 according to an embodiment of the present invention will be described below with reference to the drawings.

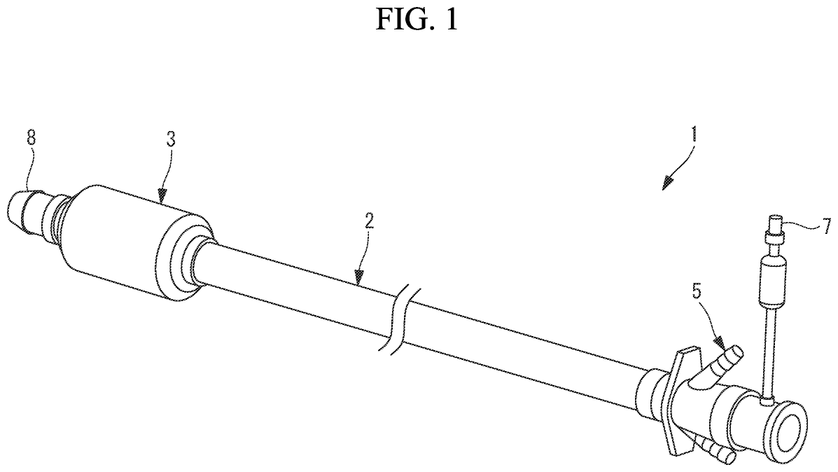

As shown in FIG. 1, the medical overtube 1 according to this embodiment includes a flexible main tube (elongated part) 2 to be inserted into a body cavity, and a balloon (fixing mechanism) 3 disposed near the distal end portion of the main tube 2.

The main tube 2 is made of, for example, silicone rubber having a Shore A hardness of 60 to 80 and having a thickness of 1 mm to 1.5 mm. Silicone rubber having a Shore A hardness of 70 and having a thickness of 1.25 mm is preferable.

The balloon 3 is made of, for example, silicone rubber having a Shore A hardness in the range of 15 to 30 and having a thickness of 0.15 mm to 0.3 mm.

The main tube 2 has a center hole (channel) 4 through which an elongated insertion section (medical device) 110 of an endoscope 100 can be inserted. The center hole 4 is open at the tip end (distal end) and the base end of the main tube 2.

A grip part 5 to be operated by an operator on the outside of the body is provided at the base end of the main tube 2. The grip part 5 is provided with a luer connector 7, to which a syringe for supplying air to the balloon 3 can be connected, and the like.

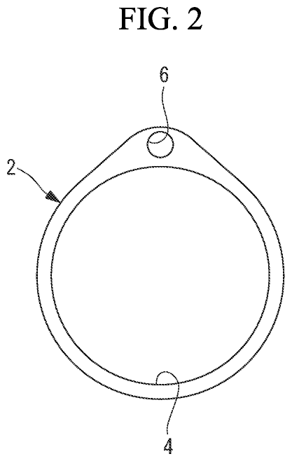

As shown in FIG. 2, the main tube 2 is provided with an air-supply lumen 6, which has a sufficiently smaller diameter than the center hole 4. The air-supply lumen 6 extends from one luer connector 7 to the position of the balloon 3, along the length direction of the main tube 2, and opens to the interior of the balloon 3. The balloon 3 can be expanded by supplying air supplied from the luer connector 7 into the balloon 3 through the air-supply lumen 6.

A tubular end tip (distal end) 8 that is made of, for example, silicone resin is provided at the distal end of the main tube 2. The main tube 2 and the end tip 8 are bonded together by a silicone adhesive.

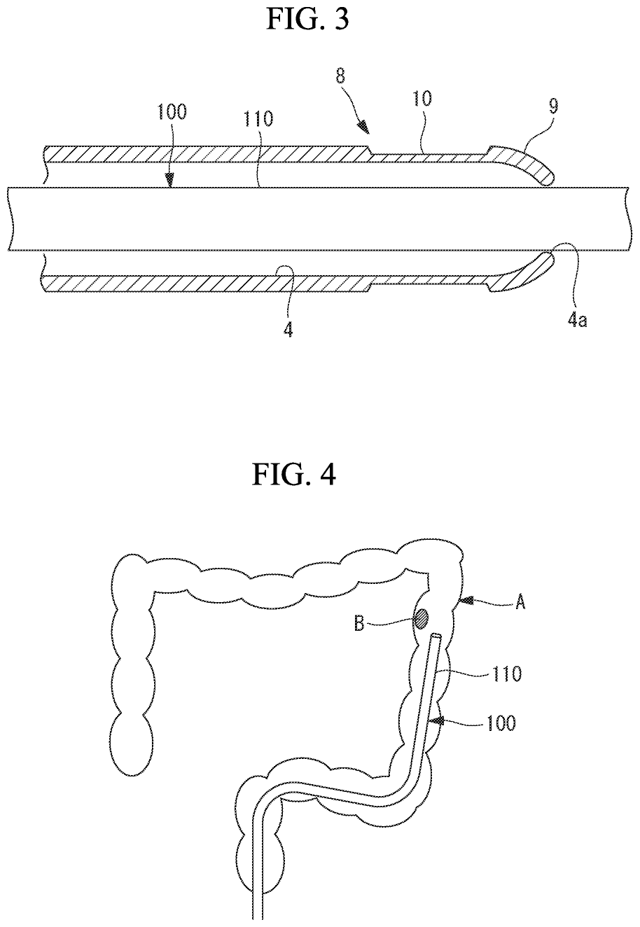

As shown in FIG. 3, the end tip 8 is made of, for example, silicone rubber having a Shore A hardness of 40 and includes a first section 9, which has an end opening 4a via which the center hole 4 opens to the outside, and a second section 10 located on the base-end side of the first section 9.

The first section 9 has a first thickness (for example, 2 mm) and is formed substantially in a ring shape that is gradually tapered toward the end opening 4a.

The inside diameter of the end opening 4a is set to be equal to or similar to the outside diameter of the insertion section 110 of the endoscope to be inserted into the center hole 4. The second section 10 is formed substantially in a ring shape having a second thickness (for example, 1 mm) that is smaller than the thickness of the first section 9.

With this configuration, the medical overtube 1 according to this embodiment satisfies Conditional Expression (1) below.

K1>K2 and K3>K2 (1)

In the Conditional Expression, K1 is the rigidity of the first section 9, K2 is the rigidity of the second section 10, and K3 is the rigidity of the main tube 2. The rigidity herein is either the flexural rigidity or the axial rigidity.

A function the thus-configured medical overtube 1 according to this embodiment will be described below.

When treating a lesion part B in a body cavity of a patient, such as the large intestine A, by using the medical overtube 1 according to this embodiment, first, as shown in FIG. 4, the insertion section 110 of the endoscope 100 is inserted into the large intestine A from the anus. Because the large intestine A is anfractuous, the insertion section 110 is inserted while the inside of the large intestine A is viewed with an endoscopic image, and is stopped at the position where the lesion part B appears in the endoscopic image.

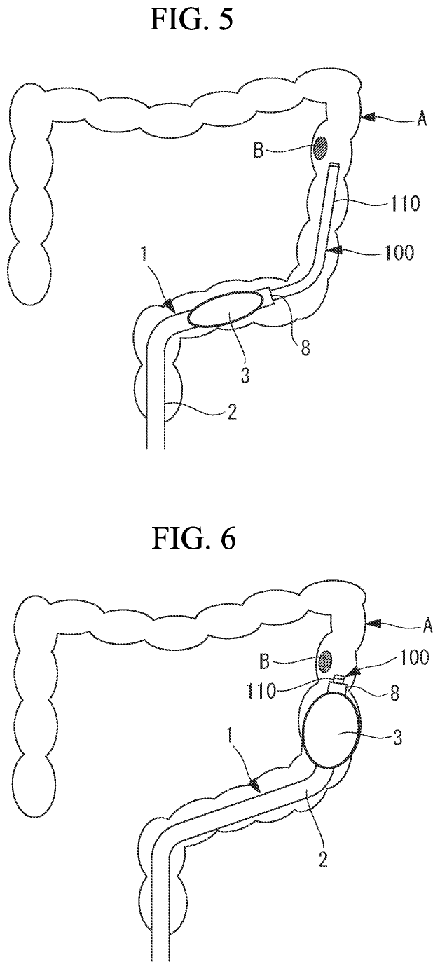

Next, as shown in FIG. 5, the medical overtube 1 with the balloon 3, which is contracted, is inserted into the large intestine A from the base-end side of the endoscope 100 by using the insertion section 110 as a guide. After that, as shown in FIG. 6, the distal end of the medical overtube 1 is inserted to the position which is slightly closer to the base-end side relative to the distal end of the insertion section 110 of the endoscope 100, and then the insertion of the medical overtube 1 is stopped.

Then, at this position, air is supplied from the luer connector 7 to expand the balloon 3 in the large intestine A and press the balloon 3 against the inner wall (body cavity inner wall) of the large intestine A to fix the distal end of the medical overtube 1 to the inside of the large intestine A.

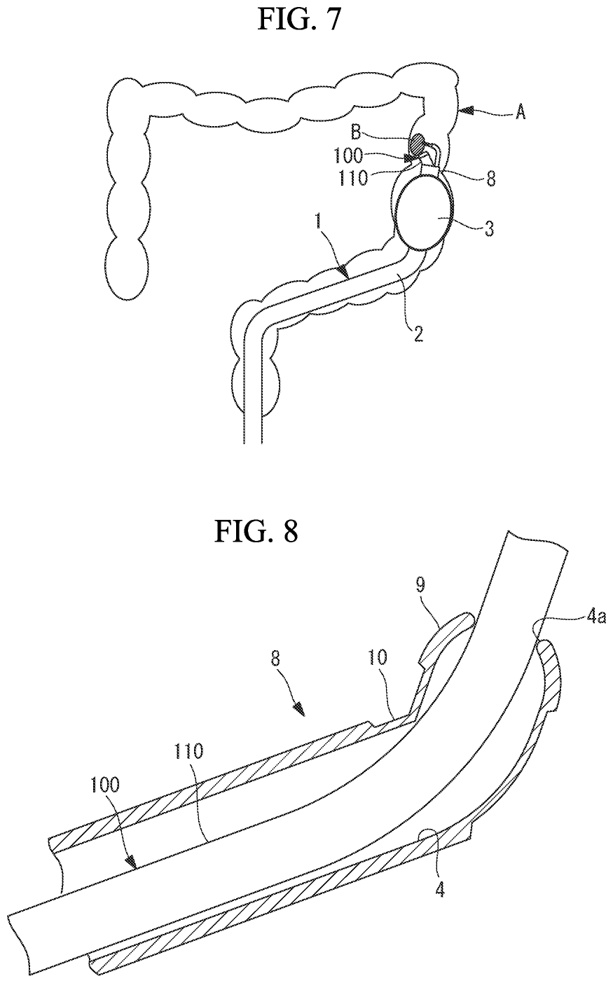

By fixing the medical overtube 1 to the large intestine A in this manner, it is possible to move the insertion section 110 of the endoscope 100 relative to the stably fixed medical overtube 1, as shown in FIG. 7. In other words, it is possible to stably operate the insertion section 110 of the endoscope 100 in the large intestine A.

In this case, the medical overtube 1 according to this embodiment is particularly effective when transitioning from the state in FIG. 6 to the state in FIG. 7. More specifically, the second section 10 has lower rigidity than the first section 9 of the end tip 8 and the main tube 2.

Hence, when the end opening 4a of the medical overtube 1 goes past a bending section of the insertion section 110 of the endoscope 100 that is bent so as to conform to the shape of the large intestine A, as shown in FIG. 8, the second section 10 deforms prior to the first section 9. As a result, the shape of the first section 9, in particular, the shape of the end opening 4a, is maintained in a shape close to the outer surface of the insertion section 110 of the endoscope 100.

In other words, because the deformation of the end opening 4a is reduced or suppressed, the gap between the end opening 4a of the medical overtube 1 and the outer surface of the insertion section 110 does not excessively increase on the outer side of the bending section of the insertion section 110 when the medical overtube 1 is advanced by using the insertion section 110 of the endoscope 100 as a guide. This leads to an advantage in that it is possible to effectively prevent the inner wall of the large intestine A from being caught in the gap between the medical overtube 1 and the outer surface of the insertion section 110 during insertion of the medical overtube 1 into the large intestine A.

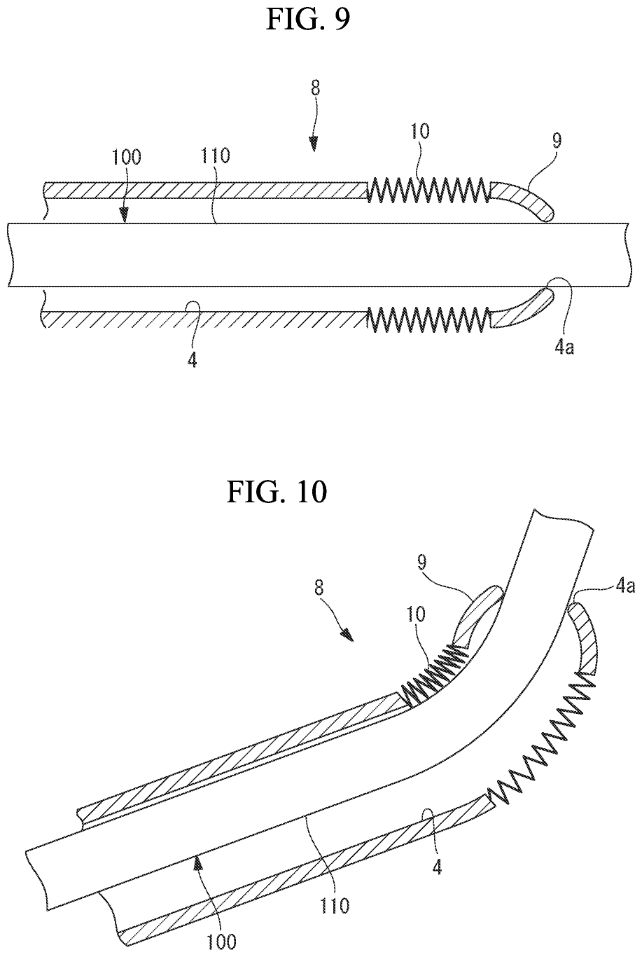

Note that, although the rigidity of the first section 9 and the rigidity of the second section 10 in the end tip 8 are differentiated by differentiating their thicknesses in this embodiment, instead of this, as shown in FIGS. 9 and 10, their rigidities may be differentiated by making the second section 10 have a bellows structure.

Furthermore, the rigidities of the first section 9 and the second section 10 may be differentiated by making them of different materials, with or without changing the thicknesses thereof.

Furthermore, in this embodiment, in addition to Conditional Expression (1), Conditional Expression (2) below may be satisfied.

K3>K1 (2)

Specifically, by making the rigidity of the main tube 2 higher than the rigidity of the first section 9, it becomes possible to prevent buckling of the main tube 2 occurring when the medical overtube 1 is pushed into the large intestine A while using the insertion section 110 as a guide.

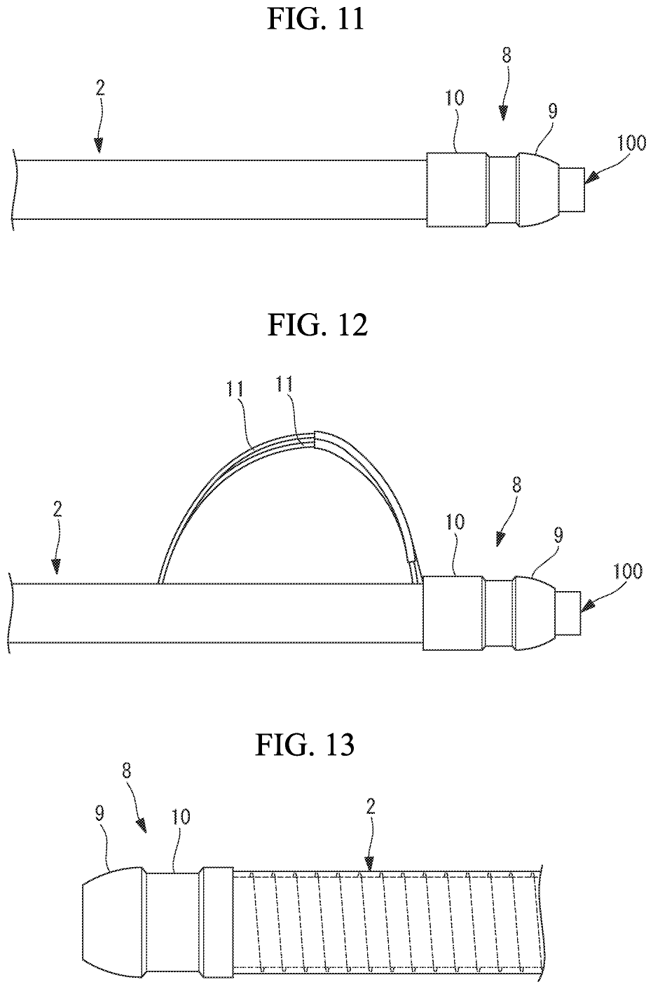

Furthermore, although the balloon 3 is used as the fixing mechanism in this embodiment, the configuration is not limited thereto. For example, as shown in FIGS. 11 and 12, it is possible to employ a fixing mechanism in which wires 11 deployed near the distal end of the main tube 2, which are pressed against the inner wall of the body cavity.

In this case, the main tube 2 is provided with, near the distal end thereof, slits extending in the longitudinal direction. The slit in the main tube 2 uses a groove-shaped mechanism, such as the ZIPLOCK mechanism, and is configured to substantially close when the wires 11 are not subjected to a compression force and to open so as to allow the curved wires 11 to project when the wires 11 are subjected to a compression force.

First, the insertion section 110 of the endoscope 100 is inserted into the body cavity of the patient. Then, the medical overtube 1 is pushed in along the inserted insertion section 110. At this time, as shown in FIG. 11, the medical overtube 1 is inserted into the body cavity with the main tube 2 accommodating the linearly extending wires 11 therein.

Once the medical overtube 1 is inserted into the body cavity, and the medical overtube 1 is positioned at a desired position, as shown in FIG. 12, a compression force is applied to the wires 11 to bend the wires 11, allowing the wires 11 to project radially outward through the slits in the main tube 2.

As a result, the curved portions of the wires 11 are pressed against the inner wall of the body cavity, and the medical overtube 1 is fixed to the inside of the body cavity.

Furthermore, when the medical overtube 1 is inserted deep into the large intestine A, the medical overtube 1 needs to be inserted through a bent portion of the large intestine A. By preventing a gap from being formed between the medical overtube 1 and the endoscope 100 by means of the end tip 8, the tissue of the large intestine A becomes less likely to be caught therein, which improves the ease of insertion of the medical overtube 1 into the body cavity.

Furthermore, as shown in FIG. 13, a medical overtube 1 with no fixing mechanism may be employed. The medical overtube 1 with no fixing mechanism will also have the same advantage.

By employing a tube with a coil as the main tube 2, it becomes possible to increase the twist rigidity and the axial rigidity while maintaining bendability.

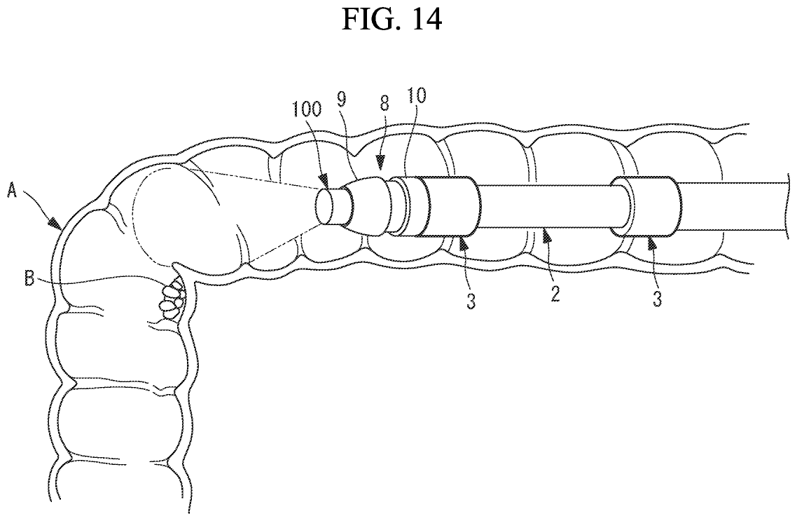

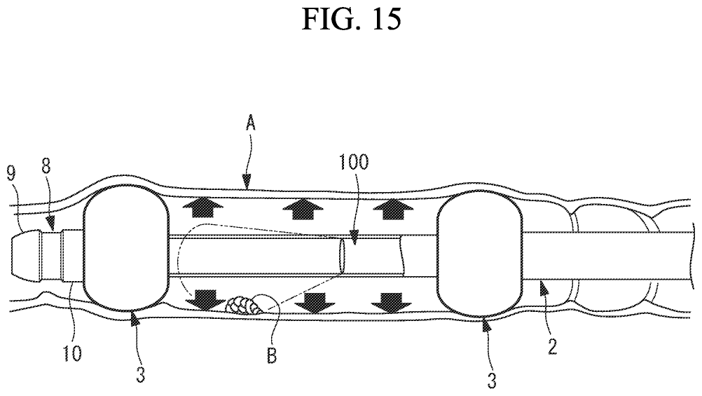

Furthermore, although the fixing mechanism with a single balloon 3 has been shown as an example in this embodiment, instead of this, as shown in FIGS. 14 and 15, a fixing mechanism with multiple balloons 3 may be employed.

In this case, one balloon 3 is disposed near the distal end of the main tube 2, and the other balloon 3 is disposed at a position distant from the one balloon 3 toward the base end.

Before the endoscope 100 is inserted into the large intestine A, the endoscope 100 is inserted into the medical overtube 1. Then, as shown in FIG. 14, the endoscope 100 and the medical overtube 1 with the balloons 3, which are contracted, are inserted into the large intestine A, and the inside of the large intestine A is viewed with an endoscopic image. At the position where the lesion part B appears in the endoscopic image, the insertion is stopped, and the other balloon 3 is expanded so as to come into contact with the inner wall of the large intestine A to expand the large intestine A.

In this state, as shown in FIG. 15, while the endoscope 100 is maintained at a position where the lesion part B appears in the endoscopic image, the medical overtube 1 alone is further inserted to the position where the lesion part B is located between the one balloon 3 and the other balloon 3, and then the one balloon 3 is expanded.

By doing so, the endoscope 100 can acquire a well visible endoscopic image through the main tube 2, which is made of a transparent material.

Furthermore, because the end tip 8 of the medical overtube 1 reduces formation of a gap occurring when the medical overtube 1 and the endoscope 100 inserted into the large intestine A are bent, the tissue of the large intestine A is less likely to be caught in the gap between the medical overtube 1 and the endoscope 100. Thus, it is possible to further improve the operability.

Although the endoscope 100 has been described as an example of the medical device in this embodiment, any other medical device, such as a treatment tool or the like, may be employed.

The following aspects are derived from the above-described embodiments.

An aspect of the present invention is a medical overtube to be inserted into a body cavity, the medical overtube including: a flexible elongated part having a channel through which an elongated medical device is inserted; and a tubular distal end portion connected to a distal end of the elongated part, wherein the distal end portion includes a first section having an end opening which forms an opening of the channel, and a second section located next to a base-end side of the first section, and the medical overtube satisfies the following Conditional Expressions:

K1>K2; and

K3>K2,

wherein K1 is rigidity of the first section, K2 is rigidity of the second section, and K3 is rigidity of the elongated part.

According to this aspect, when the medical overtube is inserted into the body cavity while using the elongated medical device inserted in the body cavity as a guide, if the medical device is bent so as to conform to the bent shape of the body cavity, the medical overtube is also bent so as to conform to the shape of the medical device. In this case, the distal end portion provided at the distal end of the elongated part having the medical device passing through the channel is bent first.

In bending the distal end portion, when the end opening in the first section constituting the distal end portion moves along the outer surface of the medical device, the second section located next to the base-end side of the first section is deformed, allowing the first section to move relatively freely. Specifically, by deforming the second section, the first section located at the distal-end side thereof can be moved along the outer surface of the medical device without being significantly deformed. This minimizes the gap formed between the end opening of the medical overtube and the medical device, thus effectively preventing the inner wall of the body cavity from being pulled into the gap.

In the above-described aspect, the medical overtube may satisfy the following Conditional Expression.

K3>K1

This makes it easy for the elongated part to transmit the compression force and the twisting force, thus improving the ease of insertion of the medical overtube.

Furthermore, in the above-described aspect, the first section and the second section may be formed of an identical kind of material, and the first section may have a greater thickness than the second section.

This makes it easy to differentiate the rigidity of the first section and the rigidity of the second section simply by differentiating the thicknesses.

Furthermore, in the above-described aspect, the second section may have a bellows structure.

This easily makes the rigidity of the first section higher than the rigidity of the second section.

Furthermore, in the above-described aspect, the first section and the second section may be made of different materials.

This easily makes the rigidity of the first section higher than the rigidity of the second section by using a composite material.

Furthermore, in the above-described aspect, the medical overtube may further include a fixing mechanism capable of being located at a base-end side relative to the distal end portion, the fixing mechanism fixing a position of the medical overtube by expanding an outside diameter of the fixing mechanism so as to be pressed against an inner wall of the body cavity.

This allows the medical overtube to be easily fixed to the inner wall of the body cavity by actuating the fixing mechanism, and thus, it is possible to move the medical device forward and backward easily inside the body cavity while using the medical overtube as a guide.

Furthermore, in the above-described aspect, the fixing mechanism may be a balloon.

This makes it possible to easily and reliably fix the medical overtube to the inner wall of the body cavity.

The aforementioned aspects provide an advantage in that it is possible to prevent the tissue from being caught in the gap between the end opening and the medical device even in a bent organ and, thus, to improve the ease of insertion.

### REFERENCE SIGNS LIST

1 medical overtube
2 main tube (elongated part)
3 balloon (fixing mechanism)
4 center hole (channel)
4a end opening
8 end tip (distal end portion)
9 first section
10 second section
11 wire (fixing structure)
110 insertion section (medical device)

1. A medical overtube to be inserted into a body cavity, the medical overtube comprising:
a flexible elongated part having a channel through which an elongated medical device is inserted; and
a tubular distal end portion connected to a distal end of the elongated part, wherein
the distal end portion includes a first section having an end opening which forms an opening of the channel, and a second section located next to a base-end side of the first section, and
the medical overtube satisfies the following Conditional Expressions:
K1>K2;
K3>K2; and
K3>K1
wherein K1 is rigidity of the first section, K2 is rigidity of the second section, and K3 is rigidity of the elongated part.

2. The medical overtube according to claim 1, wherein
the first section and the second section are formed of an identical kind of material, and
the first section has a greater thickness than the second section.

3. The medical overtube according to claim 1, wherein the second section has a bellows structure.

4. The medical overtube according to claim 1, wherein the first section and the second section are made of different materials.

5. The medical overtube according to claim 1, further comprising a fixing mechanism capable of being located at a base-end side relative to the distal end portion, the fixing mechanism fixing a position of the medical overtube by expanding an outside diameter of the fixing mechanism so as to be pressed against an inner wall of the body cavity.

6. The medical overtube according to claim 5, wherein the fixing mechanism is a balloon.

7. A medical overtube to be inserted into a body cavity, the medical overtube comprising:
a flexible elongated part having a channel through which an elongated medical device is inserted; and
a tubular distal end portion connected to a distal end of the elongated part, wherein
the distal end portion includes a first section having an end opening which forms an opening of the channel, and a second section located next to a base-end side of the first section,
the first section and the second section are formed of an identical kind of material, and
the first section has a greater thickness than the second section,
wherein the medical overtube satisfies the following Conditional Expressions:
K1>K2,
K3>K2, and
K3>K1,
wherein K1 is rigidity of the first section, K2 is rigidity of the second section, and K3 is rigidity of the elongated part.

8. The medical overtube according to claim 7, further comprising a fixing mechanism capable of being located at a base-end side relative to the distal end portion, the fixing mechanism fixing a position of the medical overtube by expanding an outside diameter of the medical overtube so as to be pressed against an inner wall of the body cavity.

9. The medical overtube according to claim 8, wherein the fixing mechanism is a balloon.
